(19) United States
(12) Patent Application Publication
(10) Pub. No.: US 2025/0134864 A1
(43) Pub. Date: May 1, 2025

(54) METHODS OF WEIGHT LOSS AND PRESERVING SKELETAL MUSCLE MASS

(71) Applicant: Rivus Pharmaceuticals, Inc., Charlottesville, VA (US)

(72) Inventors: Shaharyar Khan, Charlottesville, VA (US); Diane Jorkasky, Devon, PA (US); Francisco Portell, Charlottesville, VA (US)

(21) Appl. No.: 18/835,559

(22) PCT Filed: Feb. 6, 2023
(86) PCT No.: PCT/US2023/062077

§ 371 Date: Aug. 2, 2024

Related U.S. Application Data
(60) Provisional application No. 63/307,506, filed on Feb. 7, 2022.
(60) Provisional application No. 63/382,456, filed on Nov. 4, 2022.

(51) Int. Cl. A61K 31/4168; A61P 3/04; A61P 21/00

(52) U.S. Cl. CPC A61K 31/4168; A61P 3/04; A61P 21/00

(57) ABSTRACT

The present disclosure provides a method of preserving skeletal muscle mass during weight loss.

### CROSS-REFERENCE TO RELATED APPLICATIONS

This International PCT Application claims priority to and the benefit of U.S. Provisional Application No. 63/307,506 filed on Feb. 7, 2022 and U.S. Provisional Application No. 63/382,456, filed on Nov. 4, 2022, the contents of which are herein incorporated by reference in their entirety.

### TECHNICAL FIELD

The present disclosure relates to a method of preserving skeletal muscle mass during weigh loss.

### BACKGROUND

Obesity is a well-known risk factor for the development of many common diseases such as type 2 diabetes (T2D) and non-alcoholic fatty liver disease (NAFLD). Obesity is best viewed as any degree of excess adiposity that imparts a health risk. Weight loss is an indispensable therapy for people with obesity because it can ameliorate the risk of developing complications, such as heart disease, liver disease, pancreas disease, kidney diseases, etc. One of the major challenges to weight loss is the retention of skeletal muscle mass. The potential health benefits of bariatric surgery or diet-induced weight loss can be compromised by associated loss of lean body mass. Cava et al., “Preserving healthy Muscle during Weight Loss”, 2017 Advances in Nutrition, 8 (3): pp. 511-519; Zamboni et al. “Sarcopenic Obesity”, 2021 Sarcopnia, pp. 147-156. The risk of low muscle mass and impaired muscle function might increase the risk of further complications. Therefore, there is a great need for a method of weight loss that can also help preserving skeletal muscle mass.

The administration of chemical uncouplers of mitochondria as a means to decrease fat deposits has been a scientific goal for many years. While there are several small molecules which uncouple mitochondrial oxidative phosphorylation, the most well known is 2,4-dinitrophenol (DNP). Though DNP is known to uncouple with robust effect, it unfortunately is associated with an unacceptable high rate of significant adverse effects (J. Med. Toxicol. 2011 September; 7 (3): 205-212). These adverse effects may include hyperthermia, tachycardia, diaphoresis and tachypnoea, eventually leading to death. Being a small, highly permeable, lipophilic acid, DNP is rapidly absorbed in the stomach. The high concentration rapidly distributes and uncouples immediately, producing high levels of heat in a short period of time. Thus, DNP has a small therapeutic index and is extremely dangerous in overdose. DNP was labelled as “extremely dangerous and not fit for human consumption” by the Federal Food, Drug and Cosmetic Act of 1938. Accordingly, there is a need for uncouplers that can safely treat mitochondria-related disorders or conditions.

5-[(2,4-dinitrophenoxy)methyl]-1-methyl-2-nitro-1H-imidazole is a novel small molecule uncoupler (Compound 1). It works as a controlled metabolic accelerator (CMA). It is designed to effectively address the root cause of metabolic diseases, the accumulation of fat and sugars in the body. CMAs work to improve cellular metabolism and increase energy expenditure and calorie consumption, reducing the accumulation of fat. Using a new controlled and targeted approach, Compound 1 can increase mitochondrial proton leak, an ongoing process in the body that dissipates energy, and accounts for 20%-40% of daily calories. Compound 1 leverages a mitochondrial uncoupling mechanism to increase substrate utilization.

In recent years, the increase of incidence and prevalence of muscle wasting in the obese population has been observed. In patients with obese sarcopenia, the retention of muscle mass is critical, and the loss of muscle mass is not only counterproductive for these patients to regain metabolic health but can be a direct cause for subsequent morbidities and loss of quality of life due to falls and increase dependence on daily care. There is, henceforth, a dire need for pharmaceutical interventions that can address excess adiposity without detrimental effects on muscle mass.

### SUMMARY

In one embodiment, the present disclosure provides a method of preserving skeletal muscle mass during bodyweight reduction in a subject in need thereof, wherein the method comprises administering to the subject a therapeutically effective amount of 5-[(2,4-dinitrophenoxy)methyl]-1-methyl-2-nitro-1H-imidazole, or a pharmaceutically acceptable salt thereof.

In another embodiment, the bodyweight reduction is attributed to fat reduction.

In another embodiment, the subject suffers from obesity, excess body fat, diabetes, high blood pressure (hypertension), dyslipidemia, hypertriglyceridemia, acquired lipodystrophy, inherited lipodystrophy, partial lipodystrophy, or metabolic syndrome.

In another embodiment, the subject suffers from disorders selected from non-alcoholic fatty liver disease (NAFLD) and non-alcoholic steatohepatitis (NASH).

In another embodiment, the subject suffers from decreased muscle mass or obese sarcopenia.

In another embodiment, the subject has a body mass index greater than 28 kg/m2.

In another embodiment, the therapeutically effective amount of Compound 1 is from about 30 mg to about 1400 mg per day, from about 50 mg to about 100 mg per day, from about 150 mg to about 600 mg per day, or from 200 mg to 550 mg orally once daily.

In another embodiment, the subject experiences fat reduction greater than 5%, 10%, 20%, 30%, or 40%.

In one embodiment, the method slows the progression of obesity, hypertension, or diabetes.

### BRIEF DESCRIPTION OF THE DRAWINGS

FIG. 1 shows the Phase 2 study design flowchart.

FIG. 2 shows body fat mass changes in FAS population.

FIG. 3 shows body fat changes in the elevated HbA1c population.

FIG. 4 shows reduction of glycated albumin (percent %) in overall (FAS) population.

FIG. 5 shows the skeletal muscle mass and fat mass change in FAS population.

FIG. 6 shows the skeletal muscle mass and fat mass change in HbA1c population.

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

5-[(2,4-Dinitrophenoxy)methyl]-1-methyl-2-nitro-1H-imidazole is a novel small molecule uncoupler. It has the following structure:

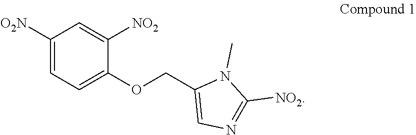 [SMILES: Cn1c(COc2ccc([N+](=O)[O-])cc2[N+](=O)[O-])cnc1[N+](=O)[O-]]

5-[(2,4-Dinitrophenoxy)methyl]-1-methyl-2-nitro-1H-imidazole may be prepared by the procedures described in WO 2018/129258.

In this disclosure, the terms Compound 1 and CM1 are interchangeable. They both refer to 5-[(2,4-Dinitrophenoxy)methyl]-1-methyl-2-nitro-1H-imidazole.

The terms “a” or “an,” as used in herein means one or more.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

As used herein, the term “about” means a range of values including the specified value, which a person of ordinary skill in the art would consider reasonably similar to the specified value. In some embodiments, the term “about” means within a standard deviation using measurements generally acceptable in the art. In some embodiments, “about” means a range extending to +/−10%, +/−5%, or +/−2% of the specified value. In some embodiments, “about” means the specified value.

As used herein, “treatment” or “treating” or “palliating” or “ameliorating” or “reducing” are used interchangeably herein. These terms refer to an approach for obtaining beneficial or desired results including but not limited to a therapeutic benefit. By therapeutic benefit means eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. Treatment includes causing the clinical symptoms of the disease to slow in development by administration of a composition; suppressing the disease, that is, causing a reduction in the clinical symptoms of the disease; inhibiting the disease, that is, arresting the development of clinical symptoms by administration of a composition after the initial appearance of symptoms; and/or relieving the disease, that is, causing the regression of clinical symptoms by administration of a composition after their initial appearance.

“Patient” or “subject” or “subject in need thereof” refers to a living organism suffering from or prone to a disease or condition that can be treated by using the methods provided herein. The term does not necessarily indicate that the subject has been diagnosed with a particular disease, but typically refers to an individual under medical supervision. Non-limiting examples include humans, other mammals.

As used herein, “administration” of a disclosed compound encompasses the delivery to a subject of a compound as described herein, or a prodrug or other pharmaceutically acceptable derivative thereof, using any suitable formulation or route of administration, e.g., as described herein.

“Pharmaceutically acceptable” refers to compounds, salts, compositions, dosage forms and other materials that are useful in preparing a pharmaceutical composition that is suitable for veterinary or human pharmaceutical use.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

An “effective amount” is an amount sufficient to accomplish a stated purpose (e.g. achieve the effect for which it is administered, treat a disease, reduce enzyme activity, reduce one or more symptoms of a disease or condition, reduce viral replication in a cell). An example of an “effective amount” is an amount sufficient to contribute to the treatment, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). Efficacy can also be expressed as “-fold” increase or decrease. For example, a therapeutically effective amount can have at least a 1.2-fold, 1.5-fold, 2-fold, 5-fold, or more effect over a control.

As used herein, the term “increase in body temperature” in a subject refers to a body temperature increase that is associated with deleterious effects on the subject, not limited to illness, physical discomfort or pain, coma and death. In one non-limiting embodiment, the significant increase in body temperature is an increase of about 0.5° C., about 1° C., about 1.5° C., about 2° C., about 2.5° C., about 3° C., about 3.5° C., about 4° C., about 4.5° C., about 5° C., about 5.5° C., about 6° C. or higher.

As used herein, HbA1c refers to hemoglobin A1c; MMRM refers to mixed model repeated measures. Negative values indicate decreases in parameter value.

### Methods of Treatment

In one aspect, disclosed herein is a method for preserving skeletal muscle mass during bodyweight reduction in a subject in need thereof, wherein the method comprises administering to the subject a therapeutically effective amount of 5-[(2,4-dinitrophenoxy)methyl]-1-methyl-2-nitro-1H-imidazole, or a pharmaceutically acceptable salt thereof.

In another aspect, disclosed here in a method for treating fibrosis, progressive fibrosis, or progressive fibrotic liver diseases NASH in a subject in need thereof, comprising administering to the subject a therapeutically effective amount of 5-[(2,4-dinitrophenoxy)methyl]-1-methyl-2-nitro-1H-imidazole, or a pharmaceutically acceptable salt thereof.

In one embodiment, the bodyweight reduction is attributed to fat reduction.

In another embodiment, the subject suffers from obesity, excess body fat, diabetes, high blood pressure (hypertension), dyslipidemia, hypertriglyceridemia, acquired lipodystrophy, inherited lipodystrophy, partial lipodystrophy, or metabolic syndrome.

In another embodiment, the subject suffers from obesity or excess body fat.

In another embodiment, the diabetes is type 2 diabetes (T2DM).

In another embodiment, the subject suffers from disorders selected from non-alcoholic fatty liver disease (NAFLD) and non-alcoholic steatohepatitis (NASH).

In another embodiment, the subject suffers from sarcopenia. Sarcopenia is an age-associated loss of muscle mass and decline in muscle strength. Increased amounts of adipose tissue often accompany sarcopenia, a condition referred to as sarcopenic obesity. Symptoms of sarcopenia can include, but are not limited to, falling, muscle weakness, slow walking speed, self-reported muscle wasting, or difficulty performing normal daily activities.

In another embodiment, the subject suffers from decreased muscle mass or sarcopenia obese.

In certain embodiments, the subject is suffering from at least one of symptoms selected from reduced exercise tolerance, fatigue, tiredness, increased time to recover after exercise, and ankle swelling.

In another embodiment, the subject has an abnormal HbA1c level.

In another embodiment, the subject has an elevated HbA1c level greater than 5.7.

In certain embodiments, the subject has a high body mass index (BMI).

In another embodiment, the subject has a body mass index greater than 28 kg/m2.

In certain embodiments, the subject's BMI is between 28.0-45.0 kg/m2.

In certain embodiments, the subject is suffering from fibrosis or progressive fibrosis.

In certain embodiments, the subject is suffering from progressive fibrotic liver diseases NASH.

In certain embodiments, the therapeutically effective amount is from about 30 mg to about 1400 mg per day, from about 100 mg to about 1000 mg per day, from about 150 mg to about 600 mg per day, from 200 mg to 550 mg orally once daily, or from about 50 mg to about 100 mg per day.

In certain embodiments, the therapeutically effective amount is about 100 mg, 150 mg, 200 mg, 250 mg, 300 mg, 350 mg, 400 mg, 450 mg, 500 mg, or 600 mg per day.

In certain embodiments, the therapeutically effective amount is about 30 mg, 35 mg, 40 mg, 45 mg, 50 mg, 55 mg, 60 mg, 65 mg, 70 mg, 75 mg, 80 mg, 85 mg, 90 mg, or 95 mg, per day.

In certain embodiments, therapeutically effective amount is about 150 mg, 300 mg, or 450 mg per day.

In another embodiment, Compound 1 is administered orally.

In another embodiment, the subject experiences fat reduction greater than 5%, 10%, 20%, or 30%.

In another embodiment, the subject with an elevated HbA1c level experiences fat reduction approximately 40%.

In another embodiment, the subject experiences at least one of:
i) a reduction of lipids by at least 10% or at least 30%;
ii) a reduction of blood pressure of at least 5 mmHg; and/or
iii) a reduction of liver fat by at least 50%.

In another embodiment, the subject experiences at least one of:
i) a reduction of lipids by at least 10% or at least 30%;
ii) a reduction of blood pressure of at least 5 mmHg; and/or
iii) a reduction of liver fat by at least 30%

In another embodiment, the method slows the progression of obesity, hypertension, or diabetes.

In one embodiment, the method slows the progression of obesity, hypertension, or diabetes.

### Pharmaceutical Dosage Forms

The present disclosure includes novel pharmaceutical dosage forms of Compound 1 or a pharmaceutically acceptable salt thereof. The dosage forms described herein are suitable for oral administration to a subject. The dosage form may be in any form suitable for oral administration, including, but not limited to, a capsule or a tablet. In some embodiments, the present disclosure provides a single unit dosage capsule or tablet form containing from about 30 mg to about 1400 mg, from about 100 mg to about 1000 mg, from about 150 mg to about 600 mg, or from 200 mg to 550 mg of Compound 1 or a pharmaceutically acceptable salt thereof. In some embodiments, Compound 1 is administered in a hydroxypropyl methylcellulose capsule.

In some embodiments, the amount of Compound 1 in a unit dosage is about 30 mg, 40 mg, 50 mg, 60 mg, 70 mg, 75 mg, 80 mg, 90 mg, 100 mg, 150 mg, 170 mg, 200 mg, 250 mg, 300 mg, 340 mg, 350 mg, 400 mg, 450 mg, 500 mg, 510 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, or 1400 mg. In some embodiments, the single unit dosage form is a capsule. In some embodiments, the single unit dosage form is a tablet

In some embodiments, the amount of Compound 1 in a unit dosage is about 30 mg, 100 mg, 200 mg, 500 mg, 600 mg, 1050 mg, or 1400 mg. In some embodiments, the amount of Compound 1 in a unit dosage is about 200 mg, 400 mg, or 550 mg. In some embodiments, the amount of Compound 1 in a unit dosage is about 170 mg, 340 mg, 510 mg. In some embodiments, the amount of Compound 1 in a unit dosage is about 150 mg, 300 mg, 450 mg.

In some embodiments, the amount of Compound 1 in a unit dosage is about 30 mg, 35 mg, 40 mg, 45 mg, 50 mg, 55 mg, 60 mg, 65 mg, 70 mg, 75 mg, 80 mg, 85 mg, 90 mg, or 95 mg, per day.

### Routes of Administration

In therapeutic use for controlling or preventing weight gain in a mammal, a compound of the present disclosure or its pharmaceutical compositions can be administered orally, or parenterally.

### Example 1: Phase 2a Study of Compound 1 in Subjects with Elevated Liver Fat and High Body Mass Index (BMI)

This was a 61-day randomized, double-blind trial placebo controlled trial to assess the safety and efficacy of 3 doses of orally administered Compound 1 compared to placebo in subjects with nonalcoholic fatty liver disease (NAFLD), elevated liver fat (>8%), and elevated body mass index (BMI) (28 to 45 kg/m2). Subjects were stratified by glycated hemoglobin (HbA1c ≥5.7%). The primary endpoint was the relative change in liver fat content from baseline to Day 61 assessed by magnetic resonance imaging proton density fat fraction (MRI-PDFF); secondary endpoints included safety, change in body composition, weight, glycemic control, and inflammation markers.

Eighty subjects were enrolled (placebo n=20, Compound 1 150 mg n=20, 300 mg n=21, 450 mg n=19). At baseline, HbA1c was elevated in 40% of subjects. At Day 61, the absolute and relative reductions in liver fat in Compound 1 treated subjects and the HbA1c subset were highly significant (p<0.0001 vs. placebo). A responder analysis using 30% or greater reduction in liver fat by MRI-PDFF showed responses in 40%, 71%, and 72% for Compound 1 150 mg, 300 mg, and 450 mg doses, respectively, and 43%, 75%, and 86% in the HbA1c subset vs. 0-5% with placebo (p<0.05 for all comparisons). Compound 1 treatment was associated with significant reductions in whole body fat, body weight, inflammatory markers, and glycated albumin. Lean body mass was preserved. No serious adverse events occurred. Six subjects discontinued early, 3 with Compound 1 (150 mg n=2, 300 n=1) and 3 with placebo. Diarrhea and flushing, most often mild, occurred in 35% and 25% of Compound 1 subjects, respectively.

### Study Design

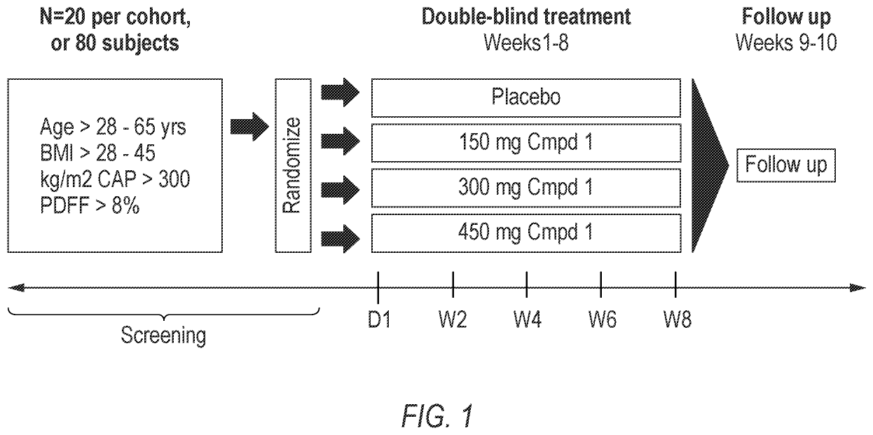

This was a Phase 2, single-center, randomized, parallel-group, double-blind, placebo-controlled, study to evaluate the safety and efficacy of Compound 1 in healthy subjects with high BMI and evidence of elevated liver fat. Subjects were screened over a 45-day period, and eligible subjects were randomized to receive once-daily oral doses of Compound 1 at 150 mg, 300 mg, or 450 mg or matching placebo under fasting conditions for 61 days. Randomization was stratified by glycated hemoglobin A1c (HbA1c) with normal baseline defined as HbA1c <5.7% and high baseline defined as HbA1c≥5.7%. A final follow-up visit occurred within 10 to 14 days after the last dose. See FIG. 1.

The randomization will be blocked and stratified by HbA_{1c}. Subjects will be stratified into tow HbA₁c strata: one subgroup of subjects with normal baseline HbA₁c defined as HbA₁c <5.7% and the other subgroup of subjects with high baseline HbA₁c defined as HbA₁c between 5.7% and 9.0% inclusive.

### Objectives

The primary efficacy and safety objectives of this study are:

### Efficacy:

To evaluate the reduction in liver fat content, as assessed by magnetic resonance imaging proton density fat fraction (MRI-PDFF), from baseline to Day 61 in subjects with elevated BMI treated with Compound 1 compared to placebo.

### Safety:

To evaluate safety and tolerability of 61 days of repeated daily dosing of Compound overweight and obese subjects as defined by BMI.
   The Secondary Objective of this Study are:
To assess the rate and amount of body weight loss after 61 days of Compound 1 treatment.
To assess change from baseline in whole body adiposity by MRI after 61 days of Compound 1 treatment.
To characterize the pharmacokinetic (PK) profile of Compound 1 and its metabolities, DNP and M1, over 61 days of dosing in subjects with high BMI.
To evaluate and correlate changes from baseline in measures of liver composition with changes in liver fat content after dosing with Compound 1.
To investigate the pharmacodynamic (PD) effects of Compound 1 on metabolic and cardiovascular risk factors.
To investigate the PD effects of Compound 1 on metabolomic, proteomic, and lipidomic profiles,
To characterize the dose/exposure relationships of the efficacy and PD effects of Compound 1, as data allow.

### Inclusion Criteria

Subjects must meet all the following inclusion criteria to be eligible:
1. Adult male or females, 28 to 65 years of age (inclusive) at the time of informed consent with BMI between 28.0 and 45.0 kg/m2 (inclusive).
   a. Female subjects of childbearing potential must be non-lactating, not pregnant as confirmed by a negative urine pregnancy test at Screening and agree to continue using an effective method of contraception for at least 4 weeks or barrier method for 2 weeks prior to first study drug administration until 30 days after the last dose of study drug.
   b. Female subjects of childbearing potential must not donate ova during the study and for at least 30 days after the last dose of study drug.
   c. Female subjects of non-childbearing potential must be surgically sterile (e.g., hysterectomy, bilateral tubal ligation, oophorectomy) or postmenopausal (no menses for >1 year with follicle stimulating hormone (FSH)>40 U/L at Screening).
   d. Male subjects who have not had a vasectomy and/or subjects who have had a vasectomy but have not had 2 post surgery negative tests for sperm must agree to use an acceptable method of contraception from time of first dose of study drug until 30 days after the last dose of the study drug, and to not donate sperm during the study and for at least 30 days after the last dose of study drug.

### Exclusion Criteria

Subjects will be excluded from the study if any of the following criteria are met:
1. Insulin-controlled diabetes.
2. Pregnant or breastfeeding or plans to become pregnant.
3. Intolerance to Magnetic Resonance Imaging (MRI) or with conditions contraindicated for MRI procedures including but not limited to inability to fit into MRI scanner or surgical clips/metallic implants/shrapnel. Subjects must not be claustrophobic, have a history of claustrophobia, or intolerance of closed or small spaces.
4. Weight gain or loss >5% in 3 months prior to study or >10% in 6 months prior to screening.
5. History of lap banding, intragastric balloon, duodenal-jejunal sleeve, or bariatric surgery within 5 years of screening, plans for bariatric surgery prior to conclusion of study participation, or plans to lose weight during this study either through a special diet, exercise program or both.
6. History of malignant hyperthermia.
7. History of chronic serious recurrent skin rashes of unknown cause.
8. History of or current clinically significant cardiovascular disease including but not limited to transient ischemic attack, stroke, cardiac arrhythmias, syncope, unstable angina, myocardial infarction in the 6 months prior to screening, congestive heart failure, or uncontrolled hypertension. (Uncontrolled hypertension is defined as a systolic blood pressure ≥160 mmHg or a diastolic blood pressure ≥100 mmHg based on an average of three resting determinations in the sitting position with an appropriately sized cuff).
9. Resting heart rate <45 or >110 bpm.
10. On screening ECG or by history:
   a. A marked baseline prolongation of QT/QTcF interval (e.g., repeated demonstration of a QTcF interval >450 msec for males and >470 msec for females).
   b. A history of additional risk factors for Torsades de Pointes (TdP) (e.g., heart failure, hypokalemia, family history of Long QT Syndrome) or a family history of sudden cardiac death of unknown origin.
11. Kidney disease, kidney transplant, or estimated glomerular filtration rate (eGFR)<50 mL/min/1.73 m2 based on the CKD-EPI Creatinine Equation (NKF 2009; https://www.kidney.org/content/ckd-epi-creatinine-equation-2009).
12. Significant lung disease requiring chronic daily medication including chronic obstructive pulmonary disease (COPD), emphysema, pulmonary fibrosis, or asthma.
13. Untreated obesity hypoventilation syndrome (OHS) or obstructive sleep apnea (OSA).
14. History of or active (acute or chronic) liver disease other than nonalcoholic fatty liver disease (NAFLD)/nonalcoholic steatohepatitis (NASH), such as but not limited to autoimmune liver disease, viral hepatitis, genetic hemochromatosis, primary biliary cirrhosis, Wilson disease, alpha-1-antitrypsin deficiency, alcohol liver disease, acute fatty liver of pregnancy or drug-induced (including acetaminophen) liver disease.
15. History of or treatment for clinically significant gastroparesis, inflammatory bowel disease, or any surgery of the upper gastrointestinal tract with the exception of cholecystectomy, or minor gastric procedures that are approved by the medical monitor.
16. History of cirrhosis and/or hepatic decompensation, including ascites, hepatic encephalopathy, or variceal bleeding.
17. History of acute pancreatitis within one year of screening or chronic pancreatitis of any cause.
18. Serum triglyceride concentrations exceeding 500 mg/dL.
19. HbA_{1c} >9.0%.
20. Familial (mother/father/sibling) and/or personal history of retinal detachment any time in the past.
21. Any history of or current diagnosis of Glaucoma.
22. Evidence of the following on screening ophthalmologic examination:
   a. Peripheral retinal pathology requiring treatment, retinal tears, or lattice that require treatment.
   b. Diabetic retinopathy with macula exudates or macula edema as shown by optical coherence tomography (OCT) and examination.
   c. Any active macular disease that affects the vision, including macula pucker (epiretinal membrane) and macular degeneration.
   d. Visually significant cataract as determined by ophthalmologist.
   e. Any previous intravitreal injection of anti-VEGF agents for macular degeneration.
   f. History of prior vitrectomy.
23. History of malignant neoplasms within 5 years of screening, except for basal cell or squamous cell skin cancer, cervical carcinoma in situ, or prostate cancer that is not currently or expected to require radiation therapy, chemotherapy and/or surgical interventions or to initiate hormonal treatment.
24. History of organ transplantation.
25. Received a COVID-19 vaccine less than 1 week prior to dosing (Visit 2/Day 1) and/or plans to receive a COVID-19 vaccine during the study period.
26. History of significant drug abuse within one year prior to Screening or frequent use of soft drugs (such as marijuana) within 3 months prior to the Screening visit, or hard drugs (such as cocaine, phencyclidine [PCP], opioid derivatives including heroin, and amphetamine derivatives) within 1 year prior to screening.
27. History of alcoholism in the last 2 years or current evidence of excessive alcohol consumption as assessed by screening evaluation using the Alcohol Use Disorders Identification Test (AUDIT, Thompson 2018), and history of regular alcohol consumption exceeding approximately 14 drinks/week for men and 7 drinks/week for women [1 drink=4 ounces (120 mL) of wine or 12 ounces (360 mL) of beer or 1 ounce (30 mL) of hard liquor] within 6 months of Screening, as determined by the Investigator.
28. Positive urine drug screen for drugs of abuse or positive phosphatidylethanol (PEth) blood test result >100 ng/ml at Screening. In instances of an exclusionary PEth value, consideration for enrollment can be provided if the principal investigator and medical monitor agree the subject's history is not consistent with alcohol abuse.
29. Current regular vaping or more than 5 cigarettes or the equivalent per week. Use of nicotine patches for smoking cessation is permitted.
30. Positive test results of hepatitis B surface antigen (HBsAg), hepatitis C virus antibody (HCV Ab), or human immunodeficiency virus (HIV1/2) antibody.
31. Neutropenia, defined as absolute neutrophil count≤1000/μL.
32. Serum AST or ALT ≥5× upper limit of normal (ULN) at screening. (One repeat test may be allowed within 7 days at the discretion of the Investigator).
33. Total bilirubin >ULN, unless due to Gilbert's syndrome or if considered normal variability in the absence of other clinically relevant liver impairment as approved by Medical Monitor.
34. International normalized ratio (INR) ≥1.3 at screening if there is other evidence of potential significant liver impairment.
35. Participation in another clinical trial at the time of screening or exposure to any investigational agent, including topical, within 30 days of screening or 5 half-lives, if half-life known.
36. No tattoo or body piercings during the course of the study. Any underlying physical or psychological medical condition that, in the opinion of the Investigator or sponsor, would make it unlikely that the subject is able comply with the study requirements or would be unable to complete the study.
37. Any condition that the investigator believes would interfere with his/her ability to provide written informed consent, comply with study instructions, or which might confound the interpretation of the study results or put the subject at undue risk.
38. Known or potential hypersensitivity to Compound 1 or its excipients.

### Prohibited Medications (Current Use):

39. Any herbal supplement, over the counter drug, mail order or prescription drug for weight loss.
40. Prescription or over the counter stimulants including: dextroamphetamine/Dexedrine, dextroamphetamine/amphetamine combination product/Adderall, or methylphenidate (Ritalin®, Concerta®).
41. Thiazolidinediones (TZD): pioglitazone/Actos, rosiglitazone/Avandia.
42. Glucagon-like peptide 1 (GLP1) agonists: exenatide/Byetta/Bydureon, lixisenatide/Adlyxin, liraglutide/Victoza, dulaglutide/Trulicity, semaglutide/Ozempic.
43. Sodium-glucose cotransporter-2 (SGLT2) inhibitors: canagliflozin/Invokana, dapagliflozin/Farxiga, empagliflozin/Jardiance, ertugliflozin/Steglatro.
44. Vitamin E: use of ursodiol or high dose vitamin E >400 IU/day for at least one month within in the last 6 months or started high dose vitamin E within last 3 months of screening.
45. Recent (within 3 months of screening) or current use of obeticholic acid/Ocaliva, systemic corticosteroids, methotrexate, tamoxifen, amiodarone, or long-term use of tetracyclines.
46. Warfarin, heparin, factor Xa inhibitors (dabigatran betrixaban edoxaban, apixaban, and rivaroxaban).
47. Concomitant medications that prolong the QT/QTc interval and are known to be associated with increased risk of Torsade des pointes as identified in the https://crediblemeds.org/website list category of ‘Known Risk’.
48. Products with cannabidiol (CBD).

### Phase 2a Trial Results

The phase 2a metabolic trial of Compound 1 was a 61-day randomized, double-blind, placebo-controlled trial designed to assess the safety and efficacy of three dose levels of Compound 1 (150 mg, 300 mg, and 450 mg) in obese participants (body mass index 28 to 45 kg/m2) with elevated liver fat (greater than 8%). Eighty (80) participants ranging in age between 28 and 65 years were randomly assigned to one of three Compound 1 treatment groups or the matched placebo group, stratified and blocked for HbA1C levels of 5.7% or greater, and dosed once daily (fasting). Participants were instructed to not change behavior with regard to diet or exercise. The Phase 2a trial met primary (liver fat reduction by MRI-PDFF) and secondary (body weight and fat reduction by abdominal MRI) endpoints. Key results and observations include:
Body weight reduction was almost exclusively from loss of fat, sparing lean body mass at all dosing levels at eight weeks, without change in diet or exercise behavior.
   Weight and fat loss was greatest at the highest dosing level, with participants losing an average of 6 pounds (p<0.001, high dose vs. placebo).
   Participants with elevated HbA1C levels experienced greater weight and fat loss, losing an average of 10 pounds (p<0.0001, high dose vs. placebo).
   Fat loss was observed in hepatic, visceral, and subcutaneous compartments by MRI.
   A >30% absolute reduction in liver fat by MRI-PDFF was observed: 40%, 71%, and 72% for the Compound 1 at 150 mg, 300 mg, and 450 mg dose levels, respectively, and 43%, 75%, and 86% with Compound 1 doses in the HbA1c subset vs. 0-5% with placebo (P<0.05 for all).
   Relative reductions in liver fat were 33%, 43%, and 40% corresponding to responder rates (>30% relative reduction) of 40%, 71% and 72% at low, mid and high doses, respectively, compared to placebo relative reduction in liver fat of 2% and responder rate of 5%.
Compound 1 was well-tolerated at all dose levels with excellent compliance. No Serious Adverse Events or deaths were reported. Diarrhea and transient flushing associated with alcohol intake, occurring in 25% and 31.6% of Compound 1 subjects respectively, were the most commonly reported Treatment Emergent Adverse Events. The majority of these events were mild; one participant discontinued Compound 1 for diarrhea in the low dose arm while no participant discontinued for any reason at the high dose.
Short-term treatment with Compound 1 was associated with significant improvements across primary and secondary endpoints related to NAFLD and obesity. The safety and tolerability of Compound 1 combined with the ability to reduce hepatic and whole-body adiposity in subjects regardless of HbA1c status suggest that long-term treatment with Compound 1 has the potential to be an effective treatment for NAFLD and other obesity associated metabolic diseases.
Compound 1 at 150 mg, 300 mg, and 450 mg demonstrated significant dose-related positive effects on the primary efficacy endpoint of the change from baseline in liver fat content by MRI-PDFF across the overall population and among those with elevated HbA1c, and these changes occurred within 61 days of treatment. The placebo-corrected change from baseline ranged from 06& to −7% with Compound 1, which compares favorably with other short-term, Phase 2 studies of drugs for NAFLD (Harrison et al, 2021; Loomba et al, 2020). Approximately 60% of the subjects overall and 68% of subjects in the subgroup experienced at least a 30% decrease in liver fat based on MRI-PDFF, and placebo-corrected mean percent change from baseline ranged from −33% to 43% for subjects overall and from −42% to −50% for subjects in the subgroup. Decreases in liver fat content were accompanied by decreases in body weight, which was accounted for by body fat without a loss of lean body mass. Improvement in liver volume, SAT, and CAP score, occurred with Compound 1, in the overall group and in the subgroup with elevated HbA1c.

At the dose of 300 mg and 450 mg doses, Compound 1 demonstrated significant positive effects on several endpoints, including InBody scale measurements of body weight, body fat mass, and percent body fat with no significant effect on skeletal muscle mass, lean body mass or dry lean mass. Compared with placebo, mean body weight decreased by 6 pounds in the 450 mg group at Day 61 and by 10 pounds in the subgroup of subjects with elevated HbA1c, while skeletal muscle mass (and lean body mass and dry lean mass) remained unchanged. Significant reductions in inflammatory and metabolic markers were observed with Compound 1. Glycated albumin was used in this study rather than HbA1c to assess metabolic control because a change in glycated albumin occurs earlier than with HbA1c (120 days) and was a better marker of glycemic control in this 61-day study. A 0.5% reduction in HbA1c was observed, in parallel with a greater reduction in glycated albumin that was statistically significant. The preferential loss of fat and improved glycemic control in the subjects with elevated HbA1c is intriguing and as longer-term therapy has the potential to improve metabolic and inflammatory health in people with type 2 diabetes and obesity.
The results indicate that treatment with Compound 1 improves the FAST score, and this improvement is of clinically relevant magnitude, evident in the lowest dose used (150 mg). The FAST score is a non-invasive test to identify patients with progressive liver fibrosis. Non-alcoholic fatty liver disease has a high prevalence, particularly in the obese and type 2 diabetic patient population. Some patients can live with elevated levels of liver fat without progressive disease, whereas others progress to develop NASH. Currently, a definitive diagnosis of NASH requires liver biopsy and histological scoring, an invasive and time-consuming diagnostic procedure associated with some risks. The challenges to identifying patients with progressive disease put patients at risk to progress to later stages of NASH without intervention. Fibroscan is a non-invasive ultrasound-based measure of liver elasticity and stiffness, providing a measure of fibrosis. Aspartate aminotransferase (AST) is a liver enzyme that can be measured in a blood sample, and elevated levels indicate liver damage. In combination, Fibroscan and AST levels (FAST score) have been shown to provide a valuable indicator of patients with progressive fibrosis (Woreta et al. PLOS One 2022 Apr. 15; 17 (4): e0266859, https://doi.org/rg 10.1371/journal.pone.0266852. A relatively small decrease in the FAST score of 0.2 points has been indicated to provide an improvement in several clinical measures in patients diagnosed with NASH (Wong et al, J. of Hepatology Vol. 75, pp. S257-S258) 2021; Newsome et al, The N England J of Medicine 2021; 384:1113-24).
Accordingly, the present disclosure provides a method for treating a subject with an elevated FAST score, wherein the subject is experiencing with progressive disease. In some embodiments, the method reduces FAST score in patients to reduce the risk of progressive disease. In some embodiments, the progressive disease includes progressive fibrosis, progress NASH, and/or progressive fibrotic liver diseases NASH.
NASH is the form of NAFLD in which you have inflammation of the liver and liver damage, in addition to fat in your liver. The inflammation and liver damage of NASH can cause fibrosis, or scarring, of the liver. NASH may lead to cirrhosis, in which the liver is scarred and permanently damaged. Our data further indicates that Compound 1 is efficacious at lower dose levels for treating patients with HbA1c levels between 5.7%-9.0% who are in progressive fibrotic liver diseases NASH.
Specific efficacy and safety of Phase 2a results are shown in Figures and Tables below:

(1) Treatment results in response (>30% liver fat reduction by MRI-PDFF) across all dose arms as shown in Table 1 below:

TABLE 1

| Responder Status | CM1 150 mg | CM1 300 mg | CM1 450 mg | All CM1 | Placebo |
|---|---|---|---|---|---|
| (FAS Population) | (N = 20) | (N = 21) | (N = 18) | (N = 59) | (N = 20) |
| Responder | 8 (40.0%) | 15 (71.4%) | 13 (72.2%) | 36 (61.0%) | 1 (5.0%) |
| Non-responder | 10 (50.0%) | 5 (23.8%) | 5 (27.8%) | 20 (33.9%) | 16 (80.0%) |

| Responder Status | 150 mg CM1 | 300 mg CM1 | 450 CM1 | All CM1 | Placebo |
|---|---|---|---|---|---|
| (HbA1c 5.7%-9.0%) | (N = 7) | (N = 8) | (N = 7) | (N = 22) | (N = 7) |
| Responder | 3 (42.9%) | 6 (75.0%) | 6 (85.7%) | 15 (68.2%) | 0 (0.0%) |
| Non-responder | 3 (42.9%) | 1 (12.5%) | 1 (14.3%) | 5 (22.74%) | 6 (85.7%) |

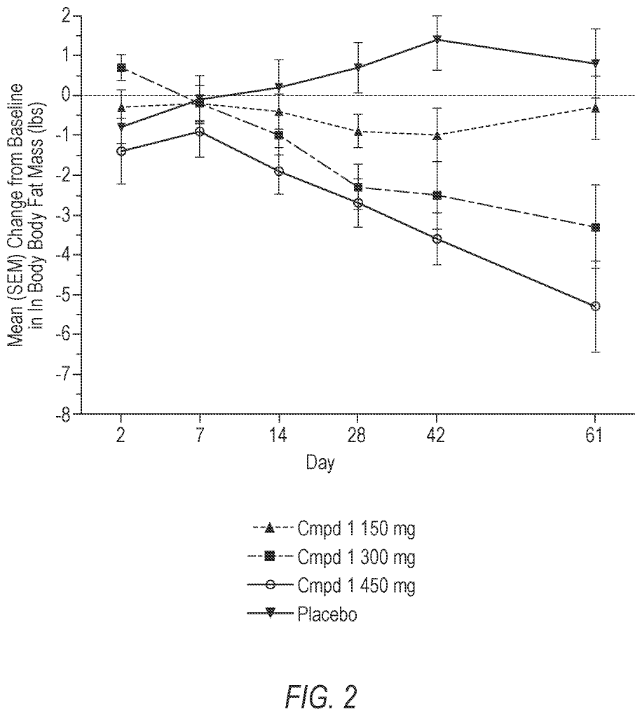

(2) Body fat loss in overall group as shown in Table 2 and FIG. 2.

TABLE 2
Summary Statistics of Observed Values and Change from
Baseline for Secondary InBody Measurements (FAS Population)
InBody Parameter: Body Fat Mass (lbs)

| Compound 1 | | 150 mg | 300 mg | 450 mg | Placebo |
|---|---|---|---|---|---|
| Visit | Statistic | (N = 20) | (N = 21) | (N = 18) | (N = 20) |
| Baseline | n | 20 | 21 | 18 | 20 |
| | Mean | 96.4 | 96.2 | 103.1 | 95.5 |
| | SD | 29.28 | 21.34 | 23.17 | 26.9 |
| | Median | 98.5 | 102.1 | 106.6 | 98.5 |
| | Min, Max | 42, 155 | 62, 133 | 53, 136 | 61, 148 |
| Day 61 | n | 18 | 20 | 18 | 17 |
| | Mean | 95.4 | 91.9 | 97.7 | 97 |
| | SD | 31.38 | 22.14 | 22.25 | 24.11 |
| | Median | 97.8 | 96.6 | 105.3 | 96.5 |
| | Min, Max | 41, 155 | 59, 132 | 49, 128 | 60, 143 |
| Change from | n | 18 | 20 | 18 | 17 |
| Baseline to | Mean | −0.3 | −3.3 | −5.3 | 0.8 |
| Day 61 | SD | 3.36 | 4.74 | 4.85 | 3.61 |
| | Median | −0.4 | −2.6 | −5.2 | 1.2 |
| | Min, Max | −6, 5 | −11, 8 | −16, 5 | −7, 6 |

Baseline is the last non-missing value prior to the first dose of study medication

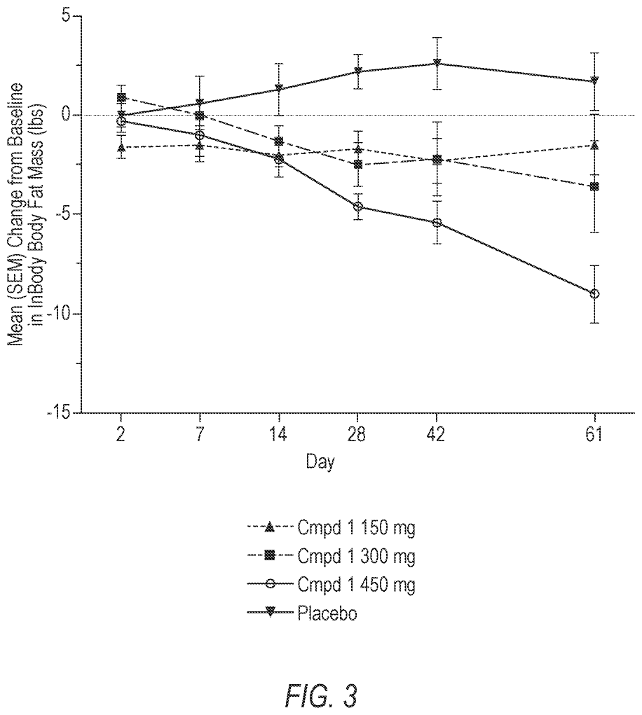

(3) Body fat change in the elevated HbA1c population (Mean±SEM) as shown in Table 3 below and FIG. 3.

TABLE 3
Summary Statistics of Observed Values and Change from Baseline
for Secondary InBody Measurements for Subjects in HbA1c 5.7%-9.0%
Subgroup Only (FAS Population)
InBody Parameter: Body Fat Mass (lbs)

| Compound 1 | | 150 mg | 00 mg | C450 mg | Placebo |
|---|---|---|---|---|---|
| Visit | Statistic | (N = 7) | (N = 8) | (N = 7) | (N = 7) |
| Baseline | n | 7 | 8 | 7 | 7 |
| | Mean | 109 | 101.9 | 106.9 | 99 |
| | SD | 20.78 | 16.49 | 30.4 | 19.73 |
| | Median | 105.3 | 107.5 | 119.1 | 103.7 |
| | Min, Max | 80, 147 | 68, 117 | 53, 136 | 61, 118 |
| Day 61 | n | 6 | 7 | 7 | 6 |
| | Mean | 107.1 | 96.2 | 97.8 | 107.1 |
| | SD | 24.02 | 19.41 | 29.05 | 11.06 |
| | Median | 102.7 | 97 | 110.2 | 107.1 |
| | Min, Max | 74, 143 | 66, 122 | 49, 128 | 93, 120 |
| Change from | n | 6 | 7 | 7 | 6 |
| Baseline to | Mean | −1.5 | −3.6 | −9 | 1.7 |
| Day 61 | SD | 3.73 | 6.11 | 3.83 | 3.56 |
| | Median | −1.8 | −5.1 | −8 | 2.2 |
| | Min, Max | −6, 5 | −11, 8 | −16, −4 | −5, 6 |

Baseline is the last non-missing value prior to the first dose of study medication

(4) Preservation of skeletal muscle mass is shown in Table 4 below:

TABLE 4
InBody Parameter: Skeletal Muscle Mass (lbs)

| | | CM1 150 | CM1 300 mg | CM1 450 mg | Placebo |
|---|---|---|---|---|---|
| Visit | Statistic | (N = 20) | (N = 21) | (N = 19) | (N = 20) |
| Change from | n | 18 | 20 | 19 | 17 |
| Baseline | Mean | −0.38 | −0.43 | −0.47 | −0.48 |
| to Day 61 | SD | 2.464 | 1.357 | 2.109 | 1.196 |
| | Median | 0.30 | −0.70 | −0.20 | −0.20 |
| | Min, Max | −7.5, 2.4 | −2.8, 2.2 | −5.3, 3.0 | −3.9, 1.1 |

| | | CM1 150 mg | CM1 300 mg | CM1 450 mg | Placebo |
|---|---|---|---|---|---|
| Visit | Statistic | (N = 7) | (N = 8) | (N = 7) | (N = 7) |
| Change from Baseline | n | 6 | 7 | 7 | 6 |
| to Day 61 | Mean | 0.63 | 0.03 | −0.03 | −0.68 |
| Elevated A1C | SD | 1.052 | 1.580 | 1.218 | 1.713 |
| | Median | 0.85 | 0.00 | 0.70 | −0.30 |
| | Min, Max | −1.3, 1.6 | −2.0, 2.2 | −1.7, 1.1 | −3.9, 1.1 |

(5) Change in MRI-Proton Density Fat Fraction (%) From Baseline to Day 61 in All Subjects and the subgroup of subjects with elevated HbA1c is shown in Table 5.

TABLE 5

| | All Subjects | | | | HbA1c 5.7% to 9.0% Subgroup | | | |
|---|---|---|---|---|---|---|---|---|
| | 150 mg | 300 mg | 450 mg | Placebo | 150 mg | 300 mg | 450 mg | Placebo |
| Statistic | (N = 20) | (N = 21) | (N = 18) | (N = 20) | (N = 7) | (N = 8) | (N = 7) | (N = 7) |

Baseline MRI-

| | | | | | | | | |
|---|---|---|---|---|---|---|---|---|
| n | 20 | 21 | 18 | 20 | 7 | 8 | 7 | 7 |
| Mean (SD) | 18.6 | 18.0 | 17.3 | 15.9 | 22.3 | 22.4 | 17.2 | 12.7 |
| Median | 16.0 | 13.9 | 13.8 | 15.0 | 22.6 | 26.0 | 14.6 | 13.8 |
| Minimum, | 9, 32 | 8, 32 | 9, 34 | 8, 27 | 9, 32 | 9, 32 | 11, 34 | 9, 16 |

Day 61

| | | | | | | | | |
|---|---|---|---|---|---|---|---|---|
| n | 18 | 20 | 18 | 17 | 6 | 7 | 7 | 6 |
| Mean (SD) | 13.7 | 11.2 | 11.2 | 16.6 | 16.2 | 14.0 | 10.7 | 15.5 |
| Median | 12.7 | 10.4 | 8.8 | 16.1 | 19.0 | 16.1 | 8.8 | 15.7 |
| Minimum, | 6, 24 | 4, 21 | 5, 24 | 7, 32 | 6, 23 | 5, 21 | 8, 22 | 8, 22 |

Day 61 − Baseline^{a}

| | | | | | | | | |
|---|---|---|---|---|---|---|---|---|
| n | 18 | 20 | 18 | 17 | 6 | 7 | 7 | 6 |
| Mean (SD) | −5.0 | −6.2 | −6.0 | 1.1 | −6.6 | −7.1 | −6.5 | 2.1 |
| | (3.09) | (3.34) | (4.12) | (3.32) | (2.69) | (3.69) | (4.13) | (2.59) |
| Median | −5.8 | −5.9 | −5.1 | 1.1 | −8.0 | −9.0 | −4.7 | 1.7 |
| Minimum, | −9, 2 | −11, 0 | −13, 2 | −5, 8 | −9, −3 | −11, 0 | −13, −3 | −1, 6 |
| maximum | | | | | | | | |
| LS Mean | −4.64 | −6.21 | −6.03 | 0.57 | −5.35 | −6.41 | −6.97 | 0.48 |
| (95% CI)^{b} | (−6.03, | (−7.52, | (−7.40, | (−0.85, | (−7.64, | (−8.48, | (−9.02, | (−1.86, |
| | −3.25) | −4.90) | −4.66) | 1.99) | −3.06) | −4.33) | −4.91) | 2.83) |

Difference in LS Mean (95% CI), Compound 1 − Placebo^{b}

| | | | | | | |
|---|---|---|---|---|---|---|
| Day 61 | −5.21 | −6.77 | −6.60 | −5.83 | −6.89 | −7.45 |
| | (−7.19, | (−8.69, | (−8.56, | (−9.26, | (−10.1, | (−10.5, |
| | − [symbol] | − [symbol] | − [symbol] | − [symbol] | − [symbol] | − [symbol] |

2-sided P-value, Compound versus Placeb

[symbol]

| | | | | | | |
|---|---|---|---|---|---|---|
| Day 61 | <.0001 | <.0001 | <.0001 | 0.0020 | 0.0002 | <.0001 |

[symbol]

indicates data missing or illegible when filed

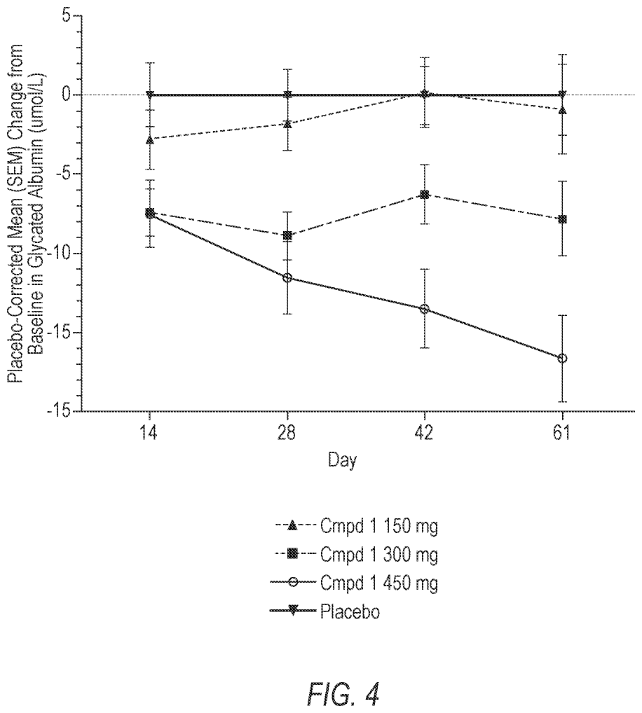

(6) Percent (%) change in glycated albumin-baseline to Day 61 as shown in Table 6 and FIG. 4.

TABLE 6
Summary Statistics of Observed Values and Change from Baseline Values
for Percent Glycated Albumin (%) by Treatment Group FAS Population

| Compound 1 | | 150 mg | 300 mg | 450 mg | Placebo |
|---|---|---|---|---|---|
| Visit | Statistic | (N = 20) | (N = 21) | (N = 18) | (N = 20) |
| Change from | n | 18 | 20 | 18 | 17 |
| Baseline to | Mean | −0.18 | −0.66 | −1.49 | 0.05 |
| Day 61 | SD | 1.282 | 0.994 | 0.958 | 0.655 |
| | Median | 0.1 | −0.55 | −1.25 | 0 |
| | Min, Max | −4.5, 1.7 | −2.5, 2.2 | −3.5, −0.2 | −0.7, 2.3 |

Baseline is the last non-missing value prior to the first dose of study medication
Summary Statistics of Observed Values and Change from Baseline Values for Percent Glycated Albumin (%) by
Treatment Group in HbA1c 5.7%-9.0% Subgroup Only (FAS Population)

| Compound 1 | | 150 mg | 300 mg | 450 mg | Placebo |
|---|---|---|---|---|---|
| Visit | Statistic | (N = 7) | (N = 8) | (N = 7) | (N = 7) |
| Change from | n | 6 | 7 | 7 | 6 |
| Baseline to | Mean | −0.78 | −0.36 | −1.73 | 0.3 |
| Day 61 | SD | 1.995 | 1.422 | 1.081 | 1.033 |
| | Median | 0.15 | −0.2 | −1.7 | 0.05 |
| | Min, Max | −4.5, 0.8 | −2.5, 2.2 | −3.5, −0.2 | −0.6, 2.3 |

Baseline is the last non-missing value prior to the first dose of study medication

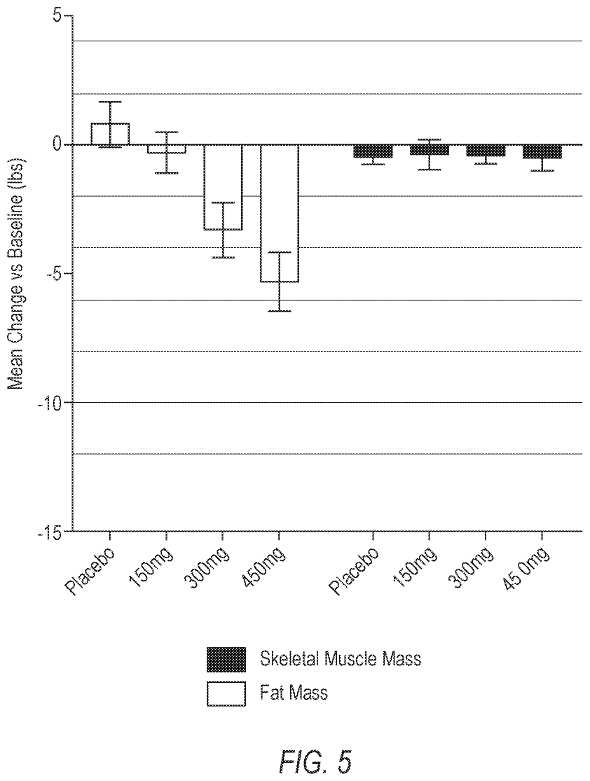

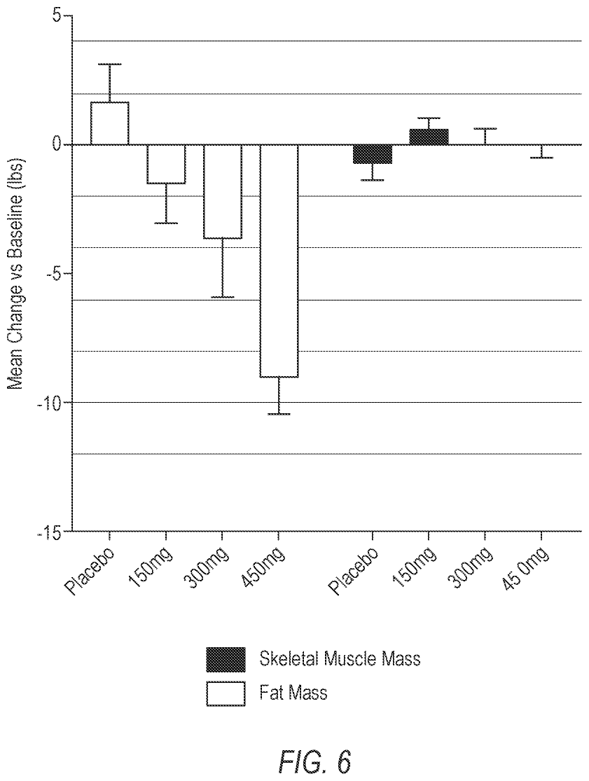

(7) MMRM Analysis of the of the change in body fat mass, percent body fat, lean body mass, and from baseline to Day 61 in all subjects and in the subgroup of subjects with elevated HbA1c are shown in FIGS. 5 and 6, and Table 7.

TABLE 7

| | All Subjects | | | | HbA1c 5.7% to 9.0% Subgroup | | | |
|---|---|---|---|---|---|---|---|---|
| | 150 mg | 300 mg | 450 mg | Placebo | 150 mg | 300 mg | 450 mg | Placebo |
| Statistic | (N = 20) | (N = 21) | (N = 18) | (N = 20) | (N = 7) | (N = 8) | (N = 7) | (N = 7) |

Body Fat Mass (pounds)

| | | | | | | | | |
|---|---|---|---|---|---|---|---|---|
| LS Mean (95% CI) | −0.42 | −3.23 | −5.34 | 0.61 | 1.78 | −3.36 | −9.02 | 1.55 |
| (Day 61 − Baseline)^{a, b} | (−2.42, | (−5.15, | (−7.37, | (−1.41, | (−5.55, | (−6.88, | (−12.54, | (−2.16, |
| | 1.58) | −1.32) | −3.32) | 2.64) | 2.00) | 0.17) | −5.50) | 5.25) |
| Difference in LS Mean | −1.03 | −3.84 | −5.95 | | −3.32 | −4.90 | −10.57 | |
| (95% CI) at Day 61 | (−3.87, | (−6.63, | (−8.81, | | (−8.61, | (−10.01, | (−15.67, | |
| (Compound 1 v. Placebo)^{b} | 1.81) | −1.06) | −3.09) | | 1.97) | 0.21) | −5.46) | |
| 2-sided P-value | 0.4715 | 0.0075 | <.0001 | | 0.2074 | 0.0593 | 0.0003 | |
| (Compound 1 v. Placebo)^{b} | | | | | | | | |

Percent Body Fat

| | | | | | | | | |
|---|---|---|---|---|---|---|---|---|
| LS Mean (95% CI) | −0.08 | −0.74 | −1.19 | 0.29 | −0.61) | −0.91 | −2.20 | 0.44 |
| (Day 61 − Baseline)^{a, b} | (−0.70, | (−1.33, | (−1.83, | (−0.34, | (−1.72, | (−1.93, | (−3.22, | (−0.62, |
| | 0.54) | −0.14) | −0.56) | 0.92) | 0.51) | 0.11) | −1.18) | 1.50) |
| Difference in LS Mean | −0.37 | −1.03 | −1.49 | | −1.05 | −1.35 | −2.64 | |
| (95% CI) at Day 61 | (−1.26, | (−1.90, | (−2.38, | | (−2.60, | (−2.82, | (−4.10, | |
| (Compound 1 v. Placebo)^{b} | 0.51) | −0.16) | −0.60) | | 0.51) | 0.12) | −1.17) | |
| 2-sided P-value | 0.4011 | 0.0207 | 0.0014 | | 0.1775 | 0.0702 | 0.0011 | |
| (Compound 1 v. Placebo | | | | | | | | |

Lean Body Mass (pounds)

| | | | | | | | | |
|---|---|---|---|---|---|---|---|---|
| LS Mean (95% CI) | −0.80 | −0.86 | −0.64 | −0.89 | 0.72 | 0.15 | −0.07 | −0.98 |
| (Day 61 − Baseline)^{a, b} | (−2.25, | (−2.26, | (−2.12, | (−2.36, | (−1.12, | (−1.51, | (−1.74, | (−2.73, |
| | 0.65) | 0.53) | 0.83) | 0.58) | 2.56) | 1.81) | 1.60) | 0.77) |
| Difference in LS Mean | 0.09 | 0.03 | 0.25 | | 1.70 | 1.13 | 0.91 | |
| (95% CI) at Day 61 | (−1.96, | (−2.00, | (−1.83, | | (−0.88, | (−1.29, | (−1.48, | |
| (Compound 1 v. Placebo)^{b} | 2.15) | 2.06) | 2.32) | | 4.29) | 3.55) | 3.31) | |
| 2-sided P-value | 0.5961 | 0.6671 | 0.5354 | | 0.1867 | 0.3439 | 0.4388 | |
| (Compound 1 v. Placebo)^{b} | | | | | | | | |

Dry Lean Mass (pounds)

| | | | | | | | | |
|---|---|---|---|---|---|---|---|---|
| LS Mean (95% CI) | −0.12 | −0.15 | −0.09 | −0.27 | 0.27 | 0.13 | 0.21 | −0.40 |
| (Day 61 − Baseline)^{a, b} | (−0.52, | (−0.54, | (−0.50, | (−0.68, | (−0.32, | (−0.41, | (−0.33, | (−0.97, |
| | 0.29) | 0.24) | 0.32) | 0.14) | 0.86) | 0.67) | 0.75) | 0.16) |
| Difference in LS Mean | 0.15 | 0.12 | 0.18 | | 0.67 | 0.54 | 0.61 | |
| (95% CI) at Day 61 | (−0.42, | (−0.44, | (−0.40, | | (−0.16, | (−0.25, | (−0.16, | |
| (Compound 1 v. Placebo)^{b} | 0.72) | 0.69) | 0.76) | | 1.50) | 1.32) | 1.39) | |
| 2-sided P-value | 0.5961 | 0.6671 | 0.5354 | | 0.1072 | 0.1694 | 0.1151 | |
| (Compound 1 v. Placebo)^{b} | | | | | | | | |

Skeletal Muscle Mass

| | | | | | | | | |
|---|---|---|---|---|---|---|---|---|
| LS Mean (95% CI) | −0.34 | −0.54 | −0.46 | −0.50 | 0.57 | −0.02 | 0.05 | −0.71 |
| (Day 61 − Baseline)^{a, b} | (−1.18, | (−1.35, | (−1.32, | (−1.35, | (−0.67, | (−1.15, | (−1.10, | (−1.89, |
| | 0.49) | 0.27) | 0.39) | 0.35) | 1.81) | 1.11) | 1.20) | 0.48) |
| Difference in LS Mean | 0.16 | −0.03 | 0.04 | | 1.28 | 0.68 | 0.76 | |
| (95% CI) at Day 61 | (−1.03, | (−1.21, | (−1.16, | | (−0.46, | (−0.96, | (−0.88, | |
| (Compound 1 v. Placebo)^{b} | 1.35) | 1.14) | 1.24) | | 3.01) | 2.32) | 2.39) | |
| 2-sided P-value | 0.7894 | 0.9540 | 0.9435 | | 0.1419 | 0.3991 | 0.3491 | |
| (Compound 1 v. Placebo)^{b} | | | | | | | | |

The LS refers to the change from baseline and the associated 95% CIs, the difference in the LS means and the associated 95% CIs, and 2-sided p-values are from an MMRM model with treatment as the fixed effect, baseline HbA1c stratification as a factor, and baseline parameter value as the covariate.

Body weight reduction in subjects administrated with Compound 1 was achieved without a corresponding loss of lean body mass, i.e. the overall observed bodyweight reduction was in large due to fat reduction whereas the treatment effect can be attributed to fat reduction entirely.

Not only was the weight loss more pronounced in participants with elevated HbA1c (all participants with an HbA1c >5.7 at baseline, amounting to approximately 40% in all cohorts) that weight loss was even more fat-specific as no reduction of muscle mass was observed from base line, and an increase of muscle mass was achieved in comparison to placebo.

In addition, 300 mg and 450 mg doses of Compound 1 demonstrated significant positive effects on several secondary endpoints, including In Body scale measurements of body weight, body fat mass, and percent body fat with no effect on skeletal muscle mass, lean body mass or dry lean mass. Compared with placebo, mean body weight decreased by 6 pounds in the 450 mg group at Day 61 and by 10 pounds in the subgroup of subjects with elevated HbA1c, while skeletal muscle mass (and lean body mass and dry lean mass) remained unchanged. Significant reductions in inflammatory and metabolic markers were observed with Compound 1. Glycated albumin was used in this study rather than HbA1c to assess metabolic control because a change in glycated albumin occurs earlier than with HbA1c (120 days) and was a better marker of glycemic control in this 61-day study. A 0.5% reduction in HbA1c was observed, in parallel with a greater reduction in glycated albumin that was statistically significant. The preferential loss of fat and improved glycemic control in the subjects with elevated HbA1c is intriguing and as longer-term therapy has the potential to improve metabolic and inflammatory health in people with type 2 diabetes and obesity.

We claim:

1. A method for preserving skeletal muscle mass during bodyweight reduction in a subject in need thereof, comprising administering to the subject a therapeutically effective amount of 5-[(2,4-dinitrophenoxy)methyl]-1-methyl-2-nitro-1H-imidazole, or a pharmaceutically acceptable salt thereof.

2. The method of claim 1 wherein the bodyweight reduction is attributed to fat reduction.

3. The method of claim 1 or 2, wherein the subject suffers from obesity, excess body fat, diabetes, high blood pressure (hypertension), dyslipidemia, hypertriglyceridemia, acquired lipodystrophy, inherited lipodystrophy, partial lipodystrophy, or metabolic syndrome.

4. The method of claim 1 or 2, wherein the subject suffers from obesity or excess body fat,

5. The method of claim 1 or 2, wherein the subject suffers from diabetes.

6. The method of claim 4, wherein the diabetes is type 2 diabetes (T2DM).

7. The method of claim 1 or 2, wherein the subject suffers from disorders selected from non-alcoholic fatty liver disease (NAFLD) and non-alcoholic steatohepatitis (NASH).

8. The method of claim 1 or 2, wherein, the subject suffers from sarcopenia.

9. The method of claim 1 or 2, wherein the subject suffers from sarcopenic obesity.

10. The method of claim 1 or 2, wherein the subject suffers from decreased muscle mass, loss of muscle mass, muscle weakness, decline in muscle strength, slow walking speed, or difficulty performing normal daily activities.

11. The method of claim 1 or 2, wherein the method slows the progression of obesity, hypertension, or diabetes.

12. The method of any one of preceding claims, wherein the subject has an abnormal HbA1c level.

13. The method of any one of preceding claims, wherein the abnormal HbA1c level is an elevated HbA1c.

14. The method of any one of preceding claims, wherein the subject has a body mass index greater than 28 kg/m2.

15. The method of any one of preceding claims, wherein the therapeutically effective amount is from about 30 mg to about 1400 mg per day, from about 100 mg to about 1000 mg per day, from about 150 mg to about 600 mg per day, from 200 mg to 550 mg per day, or from about 50 mg to about 100 mg per day.

16. The method of any one of preceding claims, wherein the therapeutically effective amount is about 100 mg, 150 mg, 200 mg, 250 mg, 300 mg, 350 mg, 400 mg, 450 mg, 500 mg, or 600 mg per day.

17. The method of any one of preceding claims, wherein the therapeutically effective amount is about 150 mg, 300 mg, or 450 mg per day.

18. The method of any one of preceding claims, wherein the therapeutically effective amount is about 30 mg, 35 mg, 40 mg, 45 mg, 50 mg, 55 mg, 60 mg, 65 mg, 70 mg, 75 mg, 80 mg, 85 mg, 90 mg, or 95 mg, per day.

19. The method of any one of preceding claims, wherein 5-[(2,4-dinitrophenoxy)methyl]-1-methyl-2-nitro-1H-imidazole is administered orally once daily.

20. The method of any one of preceding claims, wherein the subject experiences fat reduction greater than 5%, 10%, 20%, 30%, or 40%.

21. The method of claim 20, wherein the subject with an elevated HbA1c level experiences fat reduction approximately 40%.

22. The method of any one of preceding claims, wherein the subject experiences at least one of:
i) a reduction of lipids by at least 10% or at least 30%;
ii) a reduction of blood pressure of at least 5 mmHg; and/or
iii) a reduction of liver fat by at least 50%.

23. The method of any one of preceding claims, wherein the subject experiences at least one of:
i) a reduction of lipids by at least 10% or at least 30%;
ii) a reduction of blood pressure of at least 5 mmHg; and/or
iii) a reduction of liver fat by at least 30%.
